(19) United States
(12) Patent Application Publication
(10) Pub. No.: US 2008/0003292 A1
(43) Pub. Date: Jan. 3, 2008

(54) NANOPARTICLES AND METHOD FOR THE PRODUCTION THEREOF

(75) Inventors: Michael AHLERS, Eberbach (DE); Conrad COESTER, Neuhof (DE); Klaus ZWIOREK, Munchen (DE); Jan ZILLIES, Munchen (DE)

Correspondence Address: LEYDIG VOIT & MAYER, LTD, 700 THIRTEENTH ST. NW, SUITE 300, WASHINGTON, DC, 20005-3960, US

(73) Assignee: GELITA AG, Eberbach (DE)

(21) Appl. No.: 11/675,643

(22) Filed: Feb. 16, 2007

Related U.S. Application Data
(63) Continuation of application No. PCT/EP05/08954, filed on Aug. 18, 2005.

(30) Foreign Application Priority Data
Aug. 20, 2004 (DE) 10 2004 041 340.1

(51) Int. Cl. A61K 9/14; A61K 38/02; A61K 9/51; B01D 21/26; B32B 27/02

(52) U.S. Cl. 424/489; 210/632; 210/665; 210/666; 210/724; 210/725; 514/2

(57) ABSTRACT

Nanoparticles are disclosed, consisting essentially of an aqueous gelatin gel, wherein the average diameter of the nanoparticles is at the most 350 nm, the polydispersity index of the nanoparticles is less than or equal to 0.15 and wherein a gelatin is used as starting material for the production process. A process for the production of these nanoparticles is also disclosed.

### CROSS-REFERENCE TO RELATED APPLICATIONS

This application is a continuation of International Application No. PCT/EP2005/008954 filed on Aug. 18, 2005, claiming the priority of German application No. 10 2004 041 340.1 filed on Aug. 20, 2004, which are each incorporated herein by reference in their entireties.

### BACKGROUND OF THE INVENTION

The present patent application relates to nanoparticles, the use of nanoparticles for the production of medications, as well as a process for the production of nanoparticles.

Nanoparticles as carrier systems for medicinal substances have been known since the 1970s. They facilitate a targeted transport of the active substances to a desired area of the body, wherein the release takes place only at the target site (so-called drug delivery systems). At the same time, the active substance which has not yet been released is effectively shielded against metabolic influences of the body. As a result, side effects can be minimized in that the molecules of the active substance arrive predominantly and selectively at their actual target site and are less of a burden on the entire organism.

Numerous synthetic starting materials, such as, e.g., polyacrylates, polyamides, polystyrenes and cyanoacrylates, are described in the literature for the production of nanoparticles. The decisive disadvantage of these macromolecules is, however, to be seen in their poor or lacking biodegradability.

Fibronectin, various polysaccharides, albumin, collagen and gelatin are known, inter alia, as natural carrier materials degradable in the body.

A further difficulty in the case of the known nanoparticles is to be seen in their, in part, broad size distribution which is disadvantageous with a view to a uniform release and transport behavior. The size distribution of such nanoparticles can be made narrower to a certain extent due to complicated centrifugation and other separation processes but this does not lead to any satisfactory result.

The object underlying the present application is, therefore, to make biodegradable nanoparticles available which ensure a uniform and definable transport of active substances. At the same time, the object is to specify a suitable process for the production of these nanoparticles.

### BRIEF SUMMARY OF THE INVENTION

This object is accomplished in the case of the nanoparticles of the type mentioned at the outset in that they consist essentially of an aqueous gelatin gel, wherein the average diameter of the nanoparticles is at the most 350 nm and the polydispersity index of the nanoparticles is less than or equal to 0.15.

Gelatin has a number of advantages as starting material for nanoparticles. It is available in a defined composition and purity and has a relatively low, antigenic potential. Gelatin is, in addition, approved for para-oral use, inter alia, as a plasma expander.

Furthermore, the amino-acid side chains of the gelatin offer the simple possibility of modifying the surface of the nanoparticles chemically, of cross-linking the gelatin or bonding molecules of the active substance to the particles covalently.

The term “aqueous gelatin gel” is to be understood within the meaning of the present application to mean that the gelatin contained in the nanoparticles is present in a hydrated form, i.e., as a hydrocolloid. Since the nanoparticles are always surrounded by an aqueous solution during their production and use, all the specifications regarding size and polydispersity of the nanoparticles relate to this hydrated form. The determination of these parameters is brought about with the standard method of photon correlation spectroscopy (PCS) which will be described in greater detail below.

The wording “essentially consisting of” is to be understood within the meaning of the invention such that the nanoparticles consist of the aqueous gelatin gel to 95% by weight or more, preferably 97% by weight or more, even more preferred to 98% by weight or more and most preferred to 99% by weight or more.

The polydispersity index is a measure for the size distribution of the nanoparticles, wherein values between 1 (maximum dispersion) and 0 (identical size of all the particles) are theoretically possible. The low polydispersity index of the nanoparticles according to the invention of at the most 0.15 ensures a selective and controllable transport of the active substance as well as the release of the active substance at the desired target site, in particular, during the absorption of the nanoparticles by body cells.

Nanoparticles with a polydispersity index of less than or equal to 0.1 are particularly preferred.

The size of the nanoparticles is a decisive factor for their usability and can vary depending on the field of application. In many cases, nanoparticles with an average diameter of at the most 200 nm are preferred.

A further embodiment of the invention relates to nanoparticles with an average diameter of at the most 150 nm, preferably from 80 to 150 nm. These may be used by exploiting the so-called EPR effect (enhanced permeability and retention). This effect facilitates the selective treatment of tumor cells which have a greater rate of absorption than healthy cells with respect to nanoparticles of the specified size range.

An additional parameter for the size distribution of the nanoparticles is the range of variation in the diameter which is preferably at the most 20 nm above and below the average value. This range may likewise be determined by means of PCS.

The properties of the nanoparticles according to the invention may also be influenced by the molecular weight distribution of the gelatin contained therein. The proportion of low molecular gelatin is important in this connection, in particular, the proportion of gelatin with a molecular weight of less than 65 kDa, in relation to the total gelatin contained in the nanoparticles.

This proportion is preferably less than 40% by weight. A proportion of less than 30% by weight, preferably 20% by weight and less, is particularly advantageous.

In the state of the art, nanoparticles which, in addition, contain further polymeric structures (e.g. nanoparticles produced in accordance with the coacervation method, such as those described in WO 01/47501 A1) are most often described in conjunction with gelatin. The nanoparticles produced thus far from pure gelatin are either unstable or do not have the advantageous parameters described above for the selective transport of active substances with respect to particle diameter and size distribution.

In a further, preferred embodiment, the gelatin contained in the nanoparticles is cross-linked. The stability of the nanoparticles is increased considerably due to cross-linking and, in addition, the degradation behavior of the nanoparticles can be adjusted selectively as a result of the degree of cross-linking chosen. This is of advantage since different fields of application normally require defined degradation times for the nanoparticles.

It is of significance, in particular, for the cross-linking that the proportion of gelatin with a molecular weight of less than 65 kDa is less than 20% by weight.

Nanoparticles which are not cross-linked are suitable for extracorporeal, in particular, diagnostic applications, with which work can be carried out below the melting point of gelatin, e.g., at room temperature.

In contrast thereto, the cross-linked nanoparticles described above are suitable, in particular, for therapeutic applications.

The gelatin may be cross-linked chemically, e.g., by means of formaldehyde, dialdehydes, isocyanates, diisocyanates, carbodiimides or alkyl dihalides.

Alternatively, an enzymatic cross-linking, e.g., by means of transglutaminase or laccase can take place.

In a further embodiment, the nanoparticles according to the invention are dried, preferably to a water content of at the most 15% by weight.

A further embodiment of the invention relates to nanoparticles, to the surface of which a pharmaceutical agent is bonded.

In a preferred embodiment, the surface of the nanoparticles is modified chemically prior to the bonding of the active substance, e.g., by means of the reaction of free amino or carboxyl groups of the gelatin, whereby charged side chains or side chains with a new chemical functionality result.

The bonding of the pharmaceutical agent to the nanoparticles or to the chemically modified nanoparticles may be brought about by adsorption forces, by way of covalent bonds or by way of ionic bonds. For example, DNA or RNA fragments can be bonded ionically to nanoparticles, the surfaces of which are positively charged as a result of a corresponding chemical modification.

In a further embodiment, the bonding of the active substance to the nanoparticles is brought about via a spacer.

Nanoparticles described in the above may be used according to the invention, insofar as they are cross-linked, for the production of medications.

The use of the nanoparticles for intracellular drug delivery systems is especially advantageous, in particular, as carrier medium for nucleic acids or peptides.

Medications with nanoparticles according to the invention can preferably be used in gene therapy.

The present invention relates, in addition, to a process for the production of nanoparticles of the type described at the outset.

The object underlying the invention with respect to the process is accomplished in accordance with the invention in that a gelatin is used as starting material for the production process, its proportion of gelatin with a molecular weight of less than 65 kDa, in relation to the total gelatin, being at the most 40% by weight.

As a result of using such a gelatin, nanoparticles with a low polydispersity and small range of variation in the particle diameter can be produced in a simple manner, in particular, the nanoparticles according to the invention with a polydispersity index of less than or equal to 0.15.

In the case of the process according to the invention, an aqueous solution is produced first of all from such a gelatin, the pH value of this solution then being adjusted to a value below 7.0. By adding a suitable precipitating agent to this solution, a de-solvation of the dissolved gelatin takes place in the form of nanoparticles which are subsequently separated from the solution by means of a simple centrifugation. A fractionation of the nanoparticles, e.g., by means of a gradient centrifugation is not necessary since their polydispersity is already in an adequately low range as a result of the production process according to the invention.

An addition of auxiliary substances to the aqueous gelatin solution, in particular, of salts or surface active substances, such as detergents, is not necessary within the framework of the process according to the invention. Nanoparticles according to the invention are, therefore, preferably essentially free from the specified additives. The process according to the invention therefore facilitates the production of nanoparticles which consist essentially only of an aqueous gelatin gel.

As a result of the use of gelatin with the molecular weight distribution described above, the formation of stable nanoparticles is ensured. In the case of this process, gelatins with a higher low molecular proportion result, in many cases, in the formation of larger aggregates or unstable particles.

The proportion of gelatin with a molecular weight of less than 65 kDa is preferably at the most 30% by weight, most preferably at the most 20% by weight.

In a preferred embodiment of the process, the adjusted pH value of the gelatin solution is smaller than or equal to 3.0, it is preferably in the range of 1.5 to 3.0. Within this range, influence can, in part, be exerted on the average particle size via the pH value, wherein a lower pH value tends to lead to smaller nanoparticles.

In a further, preferred embodiment, acetone, alcohols, such as, e.g., ethanol are used as precipitating agents or mixtures of these precipitating agents with one another or with water, wherein acetone is preferred as precipitating agent.

As a result of the use of such volatile precipitating agents, it is possible, to a considerable degree, to avoid proportions of the precipitating agent being incorporated into and/or remaining in the nanoparticles and so they consist essentially only of the aqueous gelatin gel.

For the production of cross-linked nanoparticles, a cross-linking agent is added after the precipitating agent has been added and prior to the centrifugation. With this embodiment, the proportion of gelatin with a molecular weight of less than 65 kDa is preferably 20% by weight or less in order to counteract any agglomeration of the particles during the cross-linking. With this process, very uniform nanoparticles can be produced with a range of variation of at the most ±20 nm and a polydispersity index of at the most 0.1.

In the following, the invention will be explained in greater detail on the basis of the examples with reference to the drawings.

### BRIEF DESCRIPTION OF THE SEVERAL VIEWS OF THE DRAWINGS

FIG. 1: shows the gel permeation chromatograms of two gelatins (FIGS. 1A and 1B, respectively) which show the molecular weight distribution of the respective gelatin;

FIG. 2: shows an electron microscopic picture of nanoparticles according to the invention; and

FIG. 3: shows a size distribution of nanoparticles produced in accordance with the invention.

### DETAILED DESCRIPTION OF THE INVENTION

Determination of the Molecular Weight Distribution

The properties of the nanoparticles produced from the gelatin can be influenced, as described above, via the molecular weight distribution of this gelatin. The molecular weight distribution may be ascertained by means of gel permeation chromatography (GPC).

The determination is carried out with an HPLC system with the following components:

| | |
|---|---|
| HPLC pump: | Pharmacia 2249 |
| UV detector: | LKW 2151 |
| Separating column: | TFK 400 SWXL with precolumn |
| | (the company Tosoh Biosep GmbH) |
| Flow agent: | 1% by weight SDS, 100 mmol/l Na₂SO₄, |
| | 10 mmol/l NaH₂PO₄/NaOH pH 5.3 |

A 1% by weight gelatin solution in water is produced by swelling the gelatin for 30 minutes and subsequently dissolving it at approximately 60° C. After filtration through a 0.2 μl single use filter, 30 μl of the gelatin solution are mixed with 600 μl of flow agent and 30 μl of a 0.01% by weight benzoic acid solution. The GPC is carried out with 20 μl of this mixture at a flow rate of 0.5 ml/min and UV detection at 214 nm.

The allocation between elution volume and molecular weight is brought about by way of calibration of the system with a standard gelatin with a known molecular weight distribution. The proportion of gelatin, which is in the respective molecular weight range, can be calculated as a result of subdivision of the chromatogram into defined areas and integration of the UV detector signal.

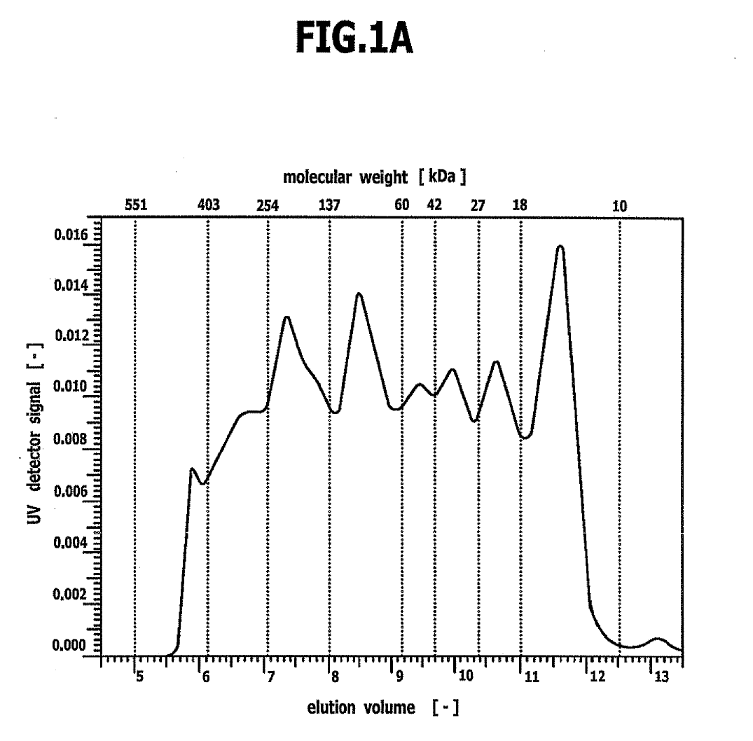

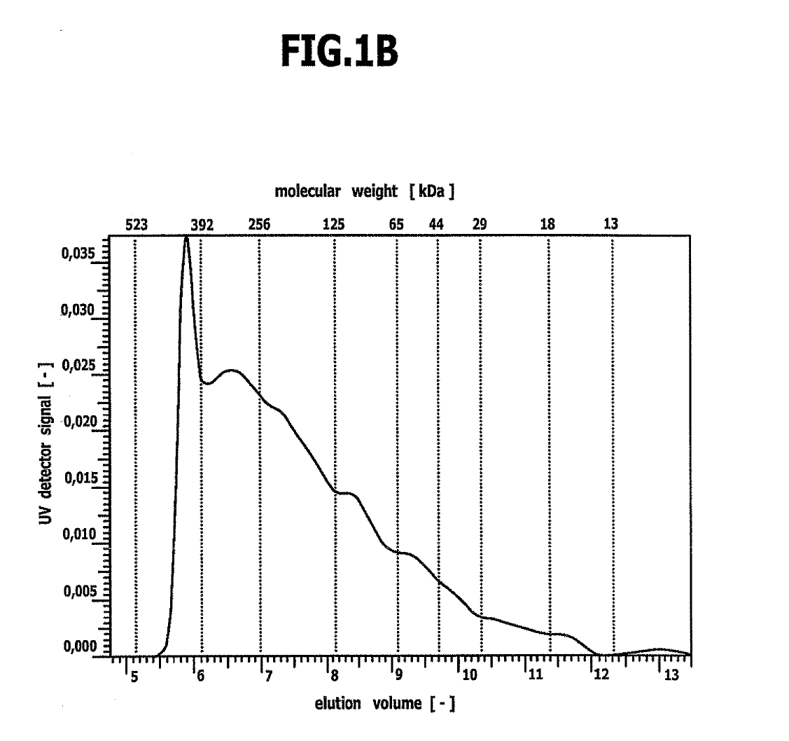

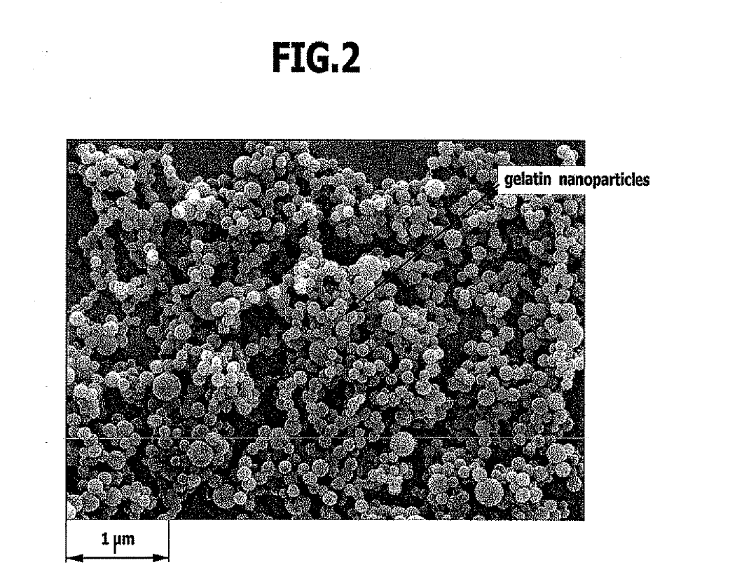

In FIG. 1, the gel permeation chromatograms of two different gelatins are illustrated by way of example.

FIG. 1A shows the GPC of a commercial pigskin gelatin (gelatin type A) with a bloom value of 175. On account of the high proportion of gelatin with a molecular weight of less than 65 kDa, which is over 45% by weight, this gelatin is not suitable for the production process for nanoparticles according to the invention and leads to particles with too high a polydispersity or to an agglomeration of the particles.

FIG. 1B shows the GPC of a pigskin gelatin with a bloom value of 310 and a proportion of gelatin with a molecular weight of less than 65 kDa of approximately 15% by weight. This gelatin is very well suited for the production process according to the invention.

Determination of the Average Particle Diameter and the Polydispersity Index

The photon correlation spectroscopy allows the determination of the average particle diameter of the nanoparticles, of the polydispersity index and of the range of variation in the particle diameter above and below the average value.

The measurements were carried out with a BI-200 goniometer version 2 (Brookhaven Instruments Corp., Holtsville, N.Y., USA). For this purpose, nanoparticle suspensions with a concentration of 10 to 50 μg/ml in demineralized water were used.

### EXAMPLE 1

This example describes the production of cross-linked nanoparticles consisting of the gelatin, the GPC of which is illustrated in FIG. 1B (with a proportion of gelatin with a molecular weight of less than 65 kDa of approximately 15% by weight).

300 mg of the said gelatin are dissolved in water at 50° C. Following the adjustment of the pH value to 2.5 with hydrogen chloride, the de-solvation of the gelatin is carried out by way of the drop by drop addition of 45 ml of acetone. After stirring for 10 minutes, 40 μl of an 8% aqueous glutaric aldehyde solution are added and, subsequently, stirred for a further 30 minutes. The nanoparticles cross-linked in this way are separated from the solution due to a 10 minute centrifugation at 10,000 g and cleansed by a three time redispersion in acetone/water (30/70). Following the last redispersion, the acetone is evaporated at 50° C.

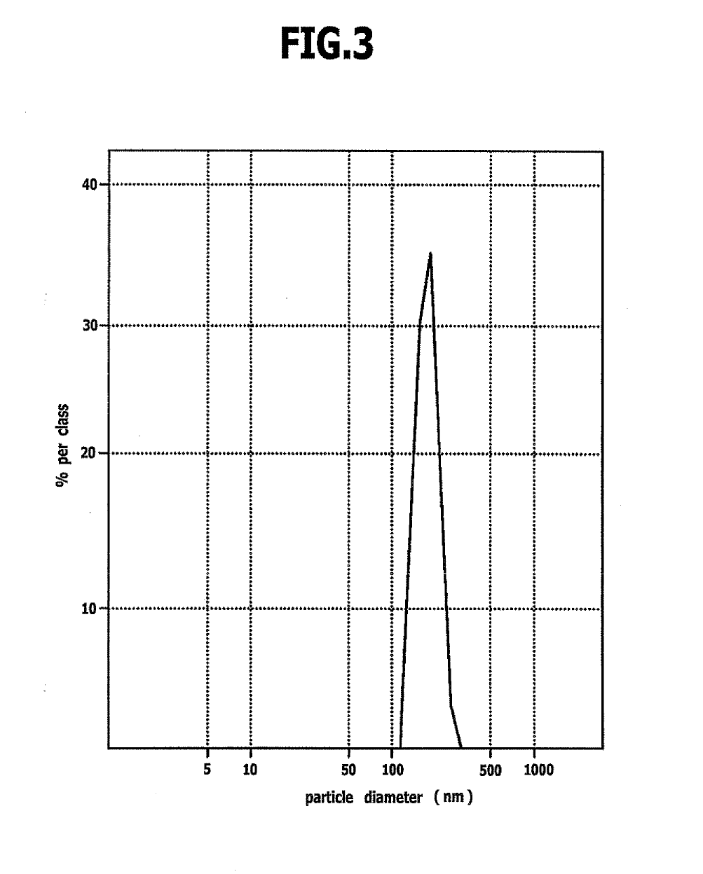

This simple process leads without additional separating steps to nanoparticles according to the invention, for which an average particle diameter of approximately 160 nm at a polydispersity index of approximately 0.08 was ascertained with the PCS method described above. The distribution of the nanoparticles according to size classes is graphically illustrated in FIG. 3.

Comparative tests on nanoparticles which are not cross-linked have resulted in the proportion of low molecular gelatin in the nanoparticles produced corresponding to a great extent to the proportion in the starting material.

### EXAMPLE 2

Cross-linked nanoparticles are produced as described in Example 1, wherein a pigskin gelatin with a bloom value of 270 is used as starting material, its proportion of gelatin with a molecular weight of less than 65 kDa being approximately 19% by weight.

An average particle diameter of approximately 173 nm at a polydispersity index of approximately 0.08 was ascertained for the nanoparticles according to the invention immediately obtained with the PCS method described above. The size distribution was comparable to the nanoparticles produced according to Example 1.

1. Nanoparticles, essentially consisting of an aqueous gelatin gel, wherein the nanoparticles have an average diameter of at most 350 nm and polydispersity index of less than or equal to 0.15.

2. The nanoparticles as defined in claim 1, wherein the polydispersity index of the nanoparticles is less than or equal to 0.1.

3. The nanoparticles as defined in claim 1, wherein the average diameter of the nanoparticles is at most 200 nm.

4. The nanoparticles as defined in claim 1, wherein the average diameter of the nanoparticles is at most 150 nm.

5. The nanoparticles as defined in claim 1, wherein the nanoparticles have a range of variation of the diameter of at most 20 nm above and below the average value.

6. The nanoparticles as defined in claim 1, wherein the proportion of gelatin with a molecular weight of less than 65 kDa, in relation to the total gelatin contained in the nanoparticles, is at most 40% by weight.

7. The nanoparticles as defined in claim 1, wherein the proportion of gelatin with a molecular weight of less than 65 kDa, in relation to the total gelatin contained in the nanoparticles, is at most 30% by weight.

8. The nanoparticles as defined in claim 1, wherein the proportion of gelatin with a molecular weight of less than 65 kDa, in relation to the total gelatin contained in the nanoparticles, is at most 20% by weight.

9. The nanoparticles as defined in claim 1, wherein the gelatin contained in the nanoparticles is cross-linked.

10. The nanoparticles as defined in claim 9, wherein the gelatin is cross-linked by formaldehyde, dialdehydes, isocyanates, diisocyanates, carbodiimides or alkyl dihalides.

11. The nanoparticles as defined in claim 9, wherein the gelatin is cross-linked enzymatically.

12. The nanoparticles as defined in claim 11, wherein the gelatin is cross-linked by transglutaminase or laccase.

13. The nanoparticles as defined in claim 1, having a water content of at most 15% by weight.

14. The nanoparticles as defined in claim 1, wherein a pharmaceutical agent is bonded to the surface of the nanoparticles.

15. The nanoparticles as defined in claim 14, wherein the bonding of the pharmaceutical agent is brought about by a chemical modification of the surface of the nanoparticles.

16. The nanoparticles as defined in claim 14, wherein the pharmaceutical agent is bonded adsorptively.

17. The nanoparticles as defined in claim 14, wherein the pharmaceutical agent is bonded covalently.

18. The nanoparticles as defined in claim 14, wherein the pharmaceutical agent is bonded ionically.

19. The nanoparticles as defined in claim 14, wherein the pharmaceutical agent is bonded via a spacer.

20. (canceled)

21. An intracellular drug delivery system comprising the nanoparticles according to claim 1, wherein the nanoparticles are a carrier medium; and, nucleic acids or peptides, carried by the carrier medium.

22. (canceled)

23. A process for the production of nanoparticles consisting essentially of an aqueous gelatin gel, comprising:
a) producing an aqueous gelatin solution, wherein the proportion of gelatin with a molecular weight of less than 65 kDa, in relation to the total gelatin, is at the most 40% by weight;
b) adjusting the pH value of the gelatin solution to a value below 7.0;
c) adding a precipitating agent and precipitating the gelatin; and
d) centrifuging the gelatin and separating the nanoparticles.

24. The process as defined in claim 23, wherein in producing the aqueous gelatin solution the proportion of gelatin with a molecular weight of less than 65 kDa, in relation to the total gelatin, is at most 30% by weight.

25. The process as defined in claim 23, wherein in producing the aqueous gelatin solution, the proportion of gelatin with a molecular weight of less than 65 kDa, in relation to the total gelatin, is at most 20% by weight.

26. The process as defined in claim 23, wherein in adjusting the pH value of the gelatin solution, the pH value is adjusted to a value smaller than or equal to 3.0.

27. The process as defined in claim 26, wherein in adjusting the pH value of the gelatin solution, the pH value is adjusted to a value in the range of 1.5 to 3.0.

28. The process as defined in claim 23, wherein in the precipitating agent is acetone, an alcohol, or a mixture of the two.

29. The process as defined in claim 23, comprising adding a cross-linking agent to the gelatin solution after precipitating the gelatin and before centrifuging the gelatin.

30. The process as defined in claim 29, wherein the cross-linking agent is selected from formaldehyde, dialdehydes, isocyanates, diisocyanates, carbodiimides or alkyl dihalides.

31. The process as defined in claim 29, wherein the cross-linking agent is an enzyme.

32. The process as defined in claim 31, wherein the cross-linking agent is laccase or transglutaminase.
